(19) United States
(12) Patent Application Publication
(10) Pub. No.: US 2013/0204636 A1
(43) Pub. Date: Aug. 8, 2013

(54) ASSOCIATION OF PROCESSING WORKFLOW GUIDELINES OF A CONTROL AND PROCESSING PROGRAM FOR MEDICAL EXAMINATION SYSTEMS

(71) Applicant: Maria Kroell, Erlangen (DE)

(72) Inventor: Maria Kroell, Erlangen (DE)

(21) Appl. No.: 13/760,494

(22) Filed: Feb. 6, 2013

(30) Foreign Application Priority Data
Feb. 6, 2012 (DE) 102012201718.6

(51) Int. Cl. G06Q 10/06; G06Q 50/22

(52) U.S. Cl. CPC G06Q 10/0633; G06Q 50/22
USPC 705/2

(57) ABSTRACT

In a method and medical examination system to associate a processing workflow guideline for multiple sub-programs of a control and processing program for processing of an examination information obtained from the medical examination apparatus, and to control an examination program at the medical examination apparatus, the examination information is transmitted in an associated tree structure with multiple hierarchy levels from the examination apparatus to a terminal apparatus, the processing workflow guideline is associated on the basis of a hierarchy level of the transmitted tree structure.

### BACKGROUND OF THE INVENTION

1. Field of the Invention

The present invention concerns: a method to associate a processing workflow guideline for multiple sub-programs of a control and processing program to process an examination information obtained from a medical examination apparatus, and to control an examination program at the medical examination apparatus, as well as a medical examination system, and a non-transitory, computer-readable data storage medium encoded with programming instructions, designed to implement such a method.

2. Description of the Prior Art

Medical examination systems often include a technical examination apparatus with a data processing device that controls a medical examination on a patient or an organism with the use of software or data processing programs. For example, different scan programs that respectively produce a defined control of the magnetic resonance scanner (MR scanner) are executed to examine a patient at the MR scanner. In an MR scanner, for example, the scan programs control the coils arranged in the MR scanner in order to acquire different examination data and examination results, for example data about a spin-spin relaxation or a spin-grid relaxation of a tissue.

The reading and evaluation of these different scan data are normally implemented at a terminal apparatus for a user with data processing functionality, at which a terminal apparatus program runs for processing and displaying the acquired scan data. For this purpose, the scan data are transferred from the MR scanner to the terminal apparatus. For example, comparisons of different examination data sets (of different scan contrasts, for example) are implemented in order to base a diagnosis on the comparison. In addition to processing of the acquired data, the terminal apparatus of the examination apparatus can be prompted to implement a selected examination.

In order to enable an evaluation of the examination data at the terminal apparatus, a number of examination data sets (i.e. results of the examination protocol) are transferred individually and serially from the examination apparatus to the terminal apparatus. At the examination apparatus, the transmitted data sets are then structured or combined into more complex data sets that then as a whole form a basis for the further evaluation and control by the control and processing program. For example, the workflow of the processing of a specific set of examination data sets at the terminal apparatus can be stored in a processing workflow guideline. The processing workflow guideline includes instructions (sub-programs, for example) as to the manner by which individual examination data sets are processed at the terminal apparatus.

However, if different examination data sets are generated after an update of the examination program at the examination apparatus, and the different examination data sets are transferred to the control and processing program, the control and processing program must be adapted in order to be able to process the new examination data sets. The manual execution of this task (i.e. an association of the transferred examination data sets with corresponding evaluation and control routines at the terminal apparatus) is complicated, expensive and time-intensive.

In addition, errors can easily occur in this procedure, that can lead to errors in a diagnosis or even to a complete inability to evaluate the examination data sets. An automatic association through a structural analysis of the examination data sets can likewise lead to incorrect results, and can be executed only with a high computing cost with a number of steps.

An object of the invention is to enable an efficient association of examination data and to increase the reliability and accuracy of the method described above.

### SUMMARY OF THE INVENTION

According to one aspect, the object according to the invention is achieved by a method to associate a processing workflow guideline for multiple sub-programs with a control and processing program (to process an examination information received from a medical examination apparatus, and to control an examination program at the medical examination apparatus), that includes the steps of a transmission of the examination information (in an associated tree structure with multiple hierarchy levels) from the examination apparatus to the terminal apparatus, and an association of the processing workflow guideline on the basis of a hierarchy level of the transmitted tree structure.

For example, the technical advantage is achieved that specific processing functions of the control and processing program can be associated at the terminal apparatus with a complete series of examination data sets at a defined hierarchy level below a node of the tree structure. The examination information that is to be used for processing is established by the data tree structure. Complicated association procedures thus can be avoided, and the reliability of the examination method and diagnosis is increased, while the number of possible medical examinations per time unit is increased.

The terminal apparatus is a data processing apparatus that is able to obtain defined output data from given input data based on a program or a processing rule. The terminal apparatus is a part of a user interface for a user via which said user can communicate with the terminal apparatus. For example, via the user interface a user can enter data into the terminal apparatus or visualize data. For example, the terminal apparatus can be a computer terminal, a workstation, a personal computer or a server.

The control and processing program is a work instruction or a series of elementary work instructions for a data processing system to achieve a defined control or processing job. The control or processing program can be composed in a programming language and can subsequently be translated into a machine code suitable for the terminal apparatus. The control job according to the invention includes a control of an examination apparatus by setting, modifying or receiving states. In particular, the control at the terminal apparatus can retrieve individual programs or sub-programs at the examination apparatus. The processing job includes the data processing of data that have been acquired by the examination apparatus.

The processing workflow guideline includes instructions of the manner by which individual examination data sets are processed at the terminal apparatus. Depending on the examination, the processing workflow guideline defines a predetermined workflow for an evaluation and processing of the acquired data, and can simultaneously serve to control the examination program at the examination apparatus. For this purpose, a number of individual control and processing routines are associated with the processing workflow guideline. The acquired data are processed in the order established by the arrangement of individual control and processing routines. A separate control and processing routine in the processing workflow guideline can be associated with every individual data set that is acquired at the examination apparatus by a sub-program of the examination program. For this purpose, for example, the processing workflow guideline can include a sub-program for every individual examination data set, which sub-program serves to evaluate the examination data acquired and transmitted by the examination apparatus. For example, the processing workflow guideline combines the control and processing of multiple sub-programs into a more complex process.

The modular design of the processing workflow guideline composed of multiple control and processing routines therefore enables individual control and processing routines to be removed, modified or added.

As used herein, a “program” is any series of instructions satisfying the rules of a programming language, which instructions can be executed at a computer or a data processing system in order to therefore provide a defined functionality.

The examination apparatus is an apparatus for the technical implementation of a medical examination. The examination apparatus includes a data processing system to control a technical workflow of the medical examination via an examination program. For example, the examination apparatus can be a computed tomography apparatus, a magnetic resonance apparatus, an x-ray apparatus, a radiation therapy apparatus, a positron emission tomography apparatus, an ultrasound apparatus or another apparatus to implement a medical treatment or examination. By executing the examination program, the examination apparatus is able to acquire medical data of a person or an organism. For example, such data can include image information or can be converted into image information.

The examination program runs on the data processing system of the examination apparatus and controls the technical workflows of the medical examination in order to acquire the desired data. In the case of an MR scanner, for example, the examination program includes programs to control electromagnetic or magnetic fields generated inside the MR scanner by coils.

The transmission of examination information includes the sending or transmission of data from the examination apparatus to the terminal apparatus, for example electronically or optically via wires or cables, or wirelessly. The examination information can be any information that is connected in any way with the examination. In the case of an MR scanner, for example, the examination information acquired by a scan program is data sets about a spin-spin relaxation or a spin-grid relaxation.

The hierarchy levels represent a system of levels that are organized above or below one another. Examination data or examination functions can be arranged at any of the hierarchy levels.

A tree structure is formed by a continuous graph that includes no cycles. The graph is maximally loop-free and minimally continuous, such that no new edges are added to the edge set without generating a loop, and no existing edge can be removed without violating the correlation property. The tree has a root element at the uppermost hierarchy level and ends at the lowest hierarchy level with the leaves.

In particular, a rooted tree (also called an out tree) as a data structure can be used as a tree structure for examination data. Given limited organization, the rooted tree can be implemented so that each node includes a fixed set of variables or an array for the references to its children. The nodes also frequently possess a reference to their parent nodes (also called back pointers). A tree of unlimited organization can be implemented in that dynamic lists are used instead of arrays. In programming languages without dynamic lists, a method has also been established in which a general tree is implemented via a binary tree for this. The tree structure that is used to transmit the examination information can be a portion (i.e. a sub-tree) of a larger tree structure at the examination apparatus.

In one embodiment, the tree structure has at least three hierarchy levels. For example, this achieves the technical advantage that a particularly simple and error-free association can be made with a fine granularity. The granularity designates the level of fineness of the grouping with which functions and activities are subdivided relative to the complete action.

In a further embodiment, the method includes the step of storing the tree structure in an object in the terminal apparatus. This achieves the technical advantage that objects with data and functions (also called methods) form the basis for an association with a processing workflow guideline. By transmitting objects with methods, not only the examination data sets but also the processing instructions belonging to the examination data sets are transmitted. These are associated with the processing workflow guideline.

An object designates an example of a specific data type or a specific class (also called an “object type”). The data structure of an object is established by the attribute or properties of its class definition. The behavior of the object is determined by the methods of the class. Classes can be derived from other classes. The class thereby inherits the data structure and the methods from the parent class.

In a further embodiment, the examination program has multiple sub-programs, and the method includes the step of an association of a sub-program with a hierarchy level of the tree structure. This achieves the technical advantage that the examination information that is obtained via the sub-program can likewise be associated with a hierarchy level. In addition, the sub-program can be selected at the terminal apparatus on the basis of a hierarchy level.

In a further embodiment, unique identification information is associated with the sub-program, and the method includes the step of an organization of the identification information in the tree structure. This achieves the technical advantage that the identification information can be evaluated at the terminal apparatus on the basis of the hierarchy level, and the data volume in the transmission is reduced.

In a further embodiment, the method includes the step of an adaptation of the control and processing program depending on the tree structure. This achieves the technical advantage that not only the processing workflow guideline but rather additionally the control is modified on the basis of the tree structure of the data, and a more precise evaluation of the data can thus be achieved.

In a further embodiment, the step of adapting the control and processing program includes the step of an adaptation of a display program to the terminal apparatus depending on the tree structure. This achieves the technical advantage that the examination data can be presented better, and the danger of misdiagnoses is thus reduced.

In a further embodiment, the method includes the step of marking specific segments of the tree structure as standard examination information. This achieves the technical advantage that the transmission volume between examination apparatuses and terminal apparatus is reduced since segments of the tree structure that include standard examination information are transmitted.

According to a further aspect, the object according to the invention is achieved by a medical examination system with an examination apparatus that is operable to obtain examination information by execution of an examination program, and a processing apparatus to control the examination apparatus by execution of a control program and to process the obtained examination information, with the examination system being configured to implement the method according to the invention. The same technical advantages can be achieved as with the method according to the invention.

The above object also is achieved in accordance with the present invention by a non-transitory, computer-readable data storage medium encoded with programming instructions that, when loaded into a computerized processor and a terminal apparatus in communication therewith, cause the processor and the terminal apparatus to execute any or all of the above-described embodiments of the method according to the invention.

### BRIEF DESCRIPTION OF THE DRAWINGS

FIG. 1 schematically illustrates a medical examination system with an MR scanner.

FIG. 2 is a view of a tree structure in accordance with the invention.

FIG. 3 is an additional view of the tree structure in accordance with the invention.

FIG. 4 is a block diagram for the association of a processing workflow guideline in accordance with the invention.

### DESCRIPTION OF THE PREFERRED EMBODIMENTS

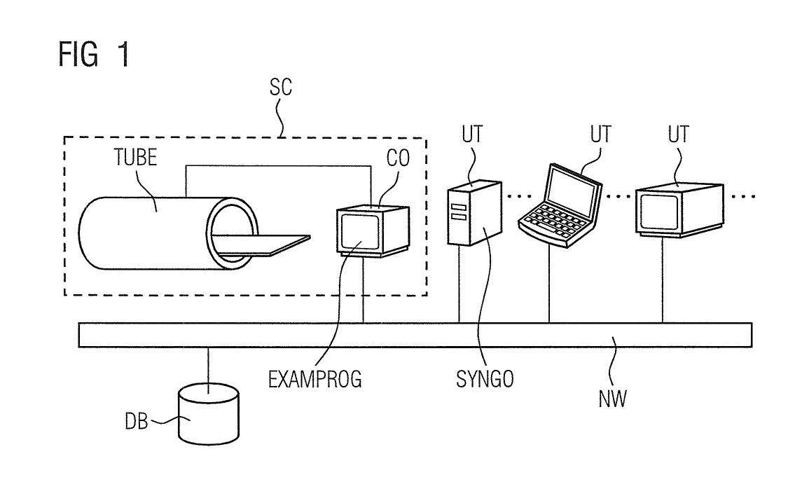

FIG. 1 shows a magnetic resonance scanner (MR scanner) SC that is representative of a medical examination apparatus, and multiple terminal apparatuses UT. Each of the terminal apparatuses UT forms at least a part of a control panel that is engaged in a data exchange with the MR scanner SC via a network NW.

To control the technical workflow of a medical examination, and to store and process acquired examination data, the magnetic resonance scanner SC has a programmable control system CO on which a control program EXAMPROG runs during an examination. For example, the control system CO can be formed by a computer, for example a workstation, a personal computer or another data processing and control unit on which software for technical implementation of the medical examination can be used. The control system CO can therefore likewise be formed by a circuit board with a processor inside the MR scanner SC.

In principle, it is possible to have the control tasks executed by multiple control systems CO, or to control multiple MR scanners SC with one control system CO. The control system CO shown in FIG. 1 has a display. In an alternative embodiment, however, the display can be omitted.

For an MR examination, suitable coil elements of an examination tube TUBE (for example) are selected by the control system CO that are individually aligned towards and arranged on the patient. These coils are activated by the control system CO via a pulse program or scan program EXAMPROG so that electromagnetic fields provided inside the MR scanner SC are generated.

In the method to control the MR scanner, a database DB can be resorted to in which data and parameters are stored that are relevant to the control of the MR scanner SC.

The control program EXAMPROG running on the control system CO, or individual sub-programs of the control program EXAMPROG, can in turn be called by the control panel at the terminal apparatus UT and the control system CO is then controlled thereby so that a user can initiate, affect and control the medical examination via said control panel. For example, the terminal apparatus UT can be a workstation, a laptop or a personal computer.

Via a control and processing program SYNGO of the terminal apparatus UT, a user can enter data for the control of the MR scanner SC that serve to control the MR scanner SC via the control system CO. In addition, the terminal apparatus UT serves to process examination data received from the MR scanner SC and prepare the examination data for a graphical presentation (for example through a Fourier transformation) so that the processed data can be displayed in an understandable manner at a display device. For example, a virtual MR scanner that simulates the respective real MR scanner SC is implemented at the terminal apparatus UT for control of said MR scanner SC.

In a medical examination, the control and processing program SYNGO at the terminal apparatus UT thus controls the examination program EXAMPROG at the MR scanner SC, which in turn controls the technical workflow of the medical examination. The examination data acquired by the MR scanner SC are then provided via the control system CO to the terminal apparatus UT and the examination data are processed by the control and processing program SYNGO.

If individual examination data sets are transmitted from the MR scanner SC to the terminal apparatus UT, the association of these examination data sets with sub-programs of the control and processing program for processing of these data sets is technically complicated. This is particularly the case when new software that generates examination data sets in a new format has been installed on the MR scanner SC.

For example, in the case of an examination of a breast, a first data set representing spin-spin relaxation and can be transmitted first from the MR scanner SC to the terminal apparatus UT, and then a second data set representing spin-grid relaxation.

For example, if—after an installation of a new examination software on the MR scanner SC—a third data set representing a correlation of spins is acquired in the same examination in addition to the two previous data sets, this third data set cannot be associated with an evaluation program.

However, if the three new data sets are transmitted in a tree structure in accordance with the invention, using the superordinate node it can easily be determined to which examination the three acquired data sets belong, namely to the examination of a breast.

The control and processing program can represent the examination data in a tree control element for association of the tree structure transmitted together with the examination information with a processing guideline. A tree control element (also called a tree view) of a graphical user interface represents a hierarchically organized list and enables a selection from the list. The illustrated tree has multiple interleaved nodes that can alternatively be unfolded or folded with a mouse click on the symbol shown before them (most often a plus sign or minus sign, or a triangle).

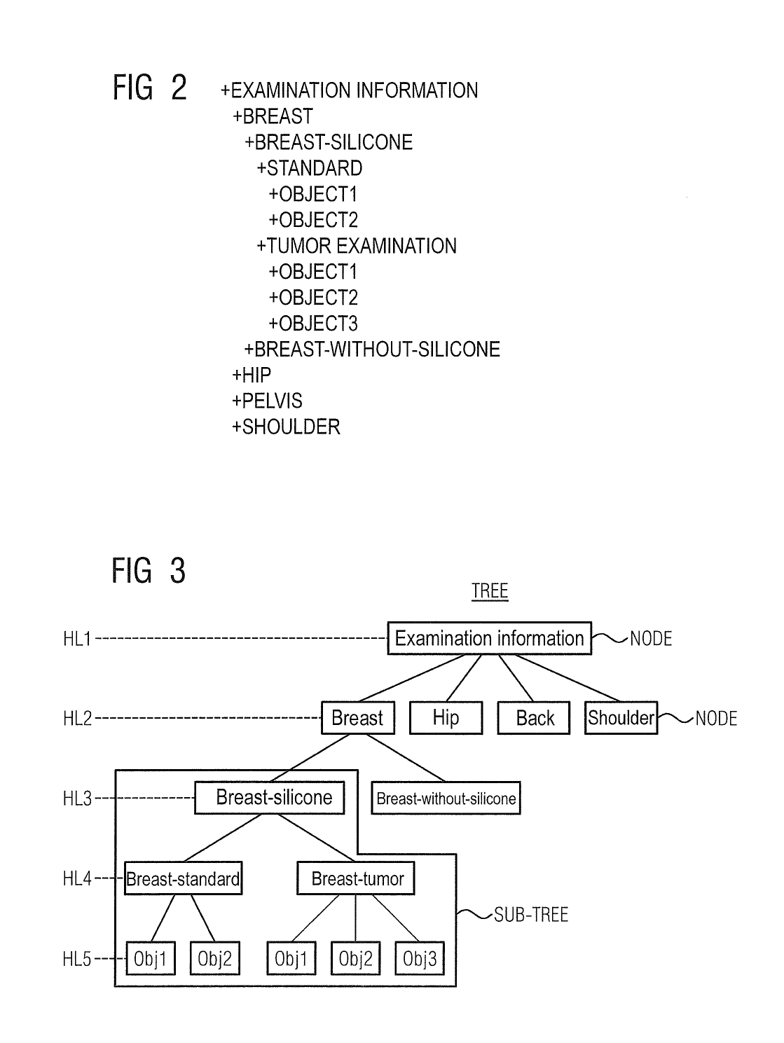

FIG. 2 shows a view of such a tree control element.

By clicking on the “+” button at the point EXAMINATION INFORMATION, the individual items of examination information of the examination apparatus can be displayed, here organized according to body regions (namely breast, hip, pelvis and shoulder). By clicking on the “+” button at the point BREAST, additional differentiations can be displayed, here organized according to properties of the breast, namely BREAST-SILICONE or BREAST-WITHOUT-SILICONE. By clicking on the “+” button at the point BREAST-SILICONE, additional differentiations can be displayed, namely STANDARD or TUMOR-EXAMINATION. Finally, by clicking on the button with regard to one of the last cited points, the individual examination information in the leaves of the tree can be retrieved, here in the form of objects, namely OBJECT1 and OBJECT2 or OBJECT1, OBJECT2 and OBJECT3.

For example, the objects OBJECT1, OBJECT2 and OBJECT3 can be DICOM objects (DICOM—Digital Imaging and Communications in Medicine). However, instead of objects the examination information can also be present in data sets, for example in lists or fields. Given MR examinations, the information acquired by a pulse program (for example a spin-grid relaxation data set, a spin-grid relaxation data set) can be stored in the leaves.

The roots of the tree are located in the upper left corner. The hierarchical structure is apparent due to the indented presentation of the individual nodes. The leaves of the tree are integrated into said tree. In another embodiment, the leaves are shown on a detail page or in a detail window, for example in the form of a list.

In general, the examination information can be arranged in a tree structure according to other suitable viewpoints, for example according to age, gender, status, body region, diagnosed illness or otherwise.

In addition to the leaves, each node of the tree can also be linked with an arbitrary item of examination information.

FIG. 3 shows another presentation of the tree structure from FIG. 2 with the node NODE. The tree structure TREE has the root element EXAMINATION at the uppermost hierarchy level HL1. The nodes BREAST, HIP, PELVIS and SHOULDER, that are logically connected with the root element, are arranged at the second hierarchy level HL2, for example. The nodes BREAST-SILICONE and BREAST-WITHOUT-SILICONE, which are connected with the superordinate node BREAST, are located at the third hierarchy level HL3. The nodes BREAST-STANDARD and BREAST-TUMOR, which are connected with the superordinate node BREAST-SILICONE, are located at the fourth hierarchy level HL4. The leaves OBJECT1, OBJECT2 and OBJECT3 at the lowermost hierarchy level HL5 represent examination information. However, examination information can be stored not only in the leaves but also in the superordinate nodes or the root element.

Such examination information can include, for example, data sets in the form of fields or lists from the medical examination, patient data or other data that have a relation to the implemented examination. Furthermore, in addition to data the examination information can also comprise executable programs or instances of objects. Moreover, it is possible that the root element, nodes or leaves of the tree structure comprise version and identification information of examination programs.

Considered independently, the sub-tree SUB-TREE represents an independent tree with three hierarchy levels and the root element BREAST-SILICONE at the uppermost hierarchy level.

The MR scanner according to the invention transmits the examination information in tree structure to the terminal apparatus. Such a transmission of the examination information does not need to comprise the entire tree of the examination information provided at the scanner; rather, it can likewise exist in that only an arbitrary sub-tree SUB-TREE is transmitted to the terminal apparatus.

It is advantageous for the examination information to include at least three hierarchy levels or overall comprises a structure adapted to the tree structure as it is present at the medical examination apparatus SC.

The tree structure of examination information that is transmitted from the MR scanner to the terminal apparatus can then be used in the control and processing program to adapt or create processing workflow guidelines. For example, at the terminal apparatus a processing workflow guideline BREAST-TUMOR with the objects OBJECT1, OBJECT2 and OBJECT3 can be formed simply by selection of the node BREAST-TUMOR from the transmitted examination information. The control and processing routines that are required for the evaluation of the examination can thereby be added to the processing workflow guideline.

This can occur through direct association of the objects OBJECT1, OBJECT2 or OBJECT3, through association of instances of the objects OBJECT1, OBJECT2 or OBJECT3 or via association of control and processing routines on the basis of the identification information with the objects OBJECT1, OBJECT2 or OBJECT3. However, in general any method can be used that utilizes the tree structure of the transmitted examination information to adapt the processing workflow guideline.

Likewise, it is in general possible to select an arbitrary node for the association of a processing workflow guideline.

The entirety of the transmitted examination information and the tree structure of the examination data can be stored in an additional object. The storage of this information in a DICOM object is particularly advantageous since this can be processed by many programs,

Furthermore, at the MR scanner SC a user can be provided with the possibility to mark one or more sub-trees as standard examination information (DEFAULT) or non-standard examination information (NON-DEFAULT). Examination information of a sub-tree that merely serves for research purposes can be marked in a simple, technical manner as non-standard examination information (NON-DEFAULT) and remain unconsidered in a transmission of the examination information to the terminal apparatus. This achieves the technical advantage that a transmission of the examination information to the terminal apparatus is accelerated.

The association of a processing workflow guideline can also be implemented based on the transmitted tree structure when the examination via the MR scanner SC has already occurred, for example based on individual examination protocols or available DICOM tags.

The control and processing program SYNGO can be expanded by a user function which allows processing workflow guidelines to be manually associated on the basis of a hierarchy level that is displayed as a table. For example, such a function can by default display two hierarchy levels. A third hierarchy level can optionally be displayed. The user interface is thereby designed to be simple and efficient.

The table is automatically filled with the exported examination information from the MR scanner. In the event that multiple MR scanners SC are connected, duplicate hierarchy entries can be masked. A user can select a node from the table and associate a processing workflow guideline with it. MR processing workflows can be selected for MR scan examination data. An access to all available processing workflows can also be optionally provided. In this method, a multiple selection can be made so that the same processing workflow guidelines can be associated with different entries in the list. It is advantageous if the entries of the list can recognize properties of the examination information.

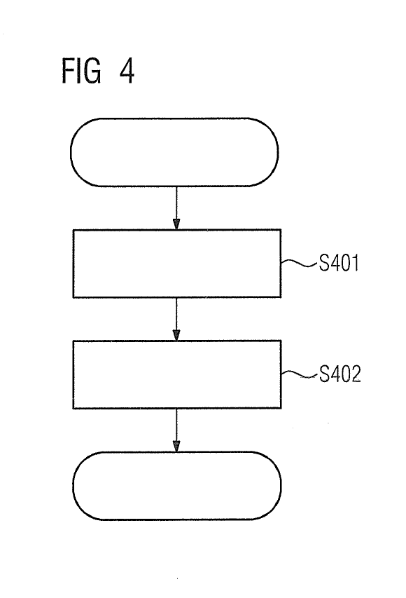

FIG. 4 shows a block diagram for the association of a processing workflow guideline.

First, in Step S401 the examination information in a tree structure with multiple hierarchy levels is transmitted from the MR scanner SC to the terminal apparatus UT. In the terminal apparatus UT, in Step S402 a processing workflow guideline is subsequently associated with the transmitted tree structure on the basis of a hierarchy level. For example, this can occur by selection of a node at a specific hierarchy level of the transmitted examination information.

Although modifications and changes may be suggested by those skilled in the art, it is the intention of the inventor to embody within the patent warranted hereon all changes and modifications as reasonably and properly come within the scope of her contribution to the art.

I claim as my invention:

1. A method to associate a processing workflow guideline for a control and processing program for processing examination information obtained from a medical examination apparatus, and to control an examination program at the medical examination apparatus, comprising:
from a computerized control unit of a medical examination apparatus, transmitting examination information, obtained by operating said medical examination apparatus with said control unit according to an examination program, in an associated tree structure with multiple hierarchy levels, to a terminal apparatus; and
at said terminal apparatus, associating a processing workflow guideline for processing said examination information, based on a hierarchy level among said multiple hierarchy levels of the transmitted tree structure.

2. A method as claimed in claim 1 comprising transmitting said examination information in an associated tree structure comprising at least three hierarchy levels.

3. A method as claimed in claim 1 comprising storing said tree structure in an object at said terminal apparatus.

4. A method as claimed in claim 1 wherein said examination program comprises multiple sub-programs, and comprising associating a respective sub-program with a respective hierarchy level of said tree structure.

5. A method as claimed in claim 4 comprising associating unique identification information with each of said sub-programs, and comprising categorizing said identification information in said tree structure.

6. A method as claimed in claim 1 comprising at said terminal apparatus, adapting said control and processing program dependent on said tree structure.

7. A method as claimed in claim 6 comprising adapting the control and processing program by adapting a display program dependent on said tree structure.

8. A method as claimed in claim 1 comprising characterizing specific segments of said tree structure as standard examination information.

9. A medical examination system to associate a processing workflow guideline for a control and processing program for processing examination information obtained from a medical examination apparatus, and to control an examination program at the medical examination apparatus, comprising:
a terminal apparatus;
a computerized control unit of a medical examination apparatus, configured to transmit examination information, obtained by operating said medical examination apparatus with said control unit according to an examination program, in an associated tree structure with multiple hierarchy levels, to said terminal apparatus; and
said terminal apparatus being configured to associate a processing workflow guideline for processing said examination information, based on a hierarchy level among said multiple hierarchy levels of the transmitted tree structure.

10. A non-transitory, computer-readable data storage medium encoded with programming instructions, said programming instructions being distributed among a computerized control unit of a medical examination apparatus and a terminal apparatus in communication with said computerized control unit, and said programming instructions causing said computerized control unit and said terminal apparatus to:
from said computerized control unit of a medical examination apparatus, transmit examination information, obtained by operating said medical examination apparatus with said control unit according to an examination program, in an associated tree structure with multiple hierarchy levels, to a terminal apparatus; and
at said terminal apparatus, associate a processing workflow guideline for processing said examination information, based on a hierarchy level among said multiple hierarchy levels of the transmitted tree structure.
